(19) United States
(12) Patent Application Publication
(10) Pub. No.: US 2025/0339254 A1
(43) Pub. Date: Nov. 6, 2025

(54) IMPLANTABLE MEDICAL DEVICE

(71) Applicant: UROMEMS, EYBENS (FR)

(72) Inventors: Hamid LAMRAOUI, VAULNAVEYS LE HAUT (FR); Gaby BOU TAYEH, GRENOBLE (FR); Lyes DJOUMI, GRENOBLE (FR)

(21) Appl. No.: 18/869,953

(22) PCT Filed: Jun. 9, 2023
(86) PCT No.: PCT/EP2023/065494

§ 371 Date: Nov. 27, 2024

(30) Foreign Application Priority Data
Jun. 9, 2022 (FR) FR2205559

(51) Int. Cl. A61F 2/00; A61F 2/26; A61F 2/48; G16H 40/63

(52) U.S. Cl. CPC A61F 2/004; A61F 2/26; A61F 2/482; G16H 40/63; A61F 2250/0002; A61F 2250/0004; A61F 2250/0081

(57) ABSTRACT

The invention relates to an implantable medical device (10) comprising:—a variable-volume fluid reservoir which is able to deform under the effect of a variation in atmospheric pressure,—an inflatable element (3) in fluid connection with the reservoir (5),—an actuator (8) which is suitable for selectively varying the volume of the reservoir (5), and—a data processing and control unit (200) which is configured to command a selective variation in the volume of the reservoir (5) by the actuator (8) and to carry out, if an atmospheric pressure estimation exceeds at least one atmospheric pressure threshold and/or if an altitude estimation exceeds at least one altitude threshold, at least one of the steps of: a) commanding. by means of the unit (200), at least one withdrawal of a volume of fluid from the inflatable element (3) to the reservoir (5) by means of the actuator (8); b) commanding, by means of the unit (200), at least one injection of a volume of fluid from the reservoir (5) to the inflatable element (3) by means of the actuator (8).

### GENERAL TECHNICAL FIELD

The present invention relates to an implantable medical device comprising a variable-volume fluid reservoir, the reservoir being able to deform under the influence of an atmospheric pressure variation.

### PRIOR ART

Some medical devices appear in the form of systems that are implantable in the body of a human or animal individual. In particular, such devices can correspond to artificial urinary sphincters used for combating urinary incontinence, gastric bands or rings suitable for restraining the stomach for the purpose of combatting obesity, inflatable penile implants used for erectile prostheses, etc.

In a manner known per se, the implantable system can function hydraulically and can comprise in particular a variable-volume fluid reservoir and an inflatable element containing a variable volume of fluid. The inflatable element is in fluid connection with the variable-volume fluid reservoir, so as to be able to transfer fluid from the reservoir to the inflatable element, and vice versa.

In the case of an occlusive implantable system, such as an artificial urinary sphincter, the inflatable element is an inflatable occlusive cuff capable of selectively blocking an anatomical passage such as a urethra in a man, or a bladder neck in a woman. Fluid can be transferred from the reservoir to the cuff to increase the pressure exerted on the passage, and conversely from the cuff to the reservoir to reduce the pressure exerted on the passage. Thus, depending on the volume of fluid in the cuff, a greater or lesser pressure can be exerted on the anatomical passage to be blocked.

Elements of the implantable fluid system can be sensitive to variations in atmospheric pressure (due for example to variations of altitude) and deform. For example, the reservoir of the implantable fluid system can comprise an elastically deformable portion which is deformed depending on atmospheric pressure variations. This deformation can cause an injection of fluid into the cuff or an uncontrolled withdrawal of fluid from the cuff and therefore an increase or a reduction in the pressure in the cuff.

Yet such pressure variations in the inflatable element are to be avoided. In the case of artificial urinary sphincters, overpressures are likely to degrade the tissues around which the cuff is arranged, for example by causing lesions in these tissues. It is therefore necessary to compensate these overpressures in order to limit the corresponding risk of degradation of the tissues.

### DISCLOSURE OF THE INVENTION

One object of the invention is to compensate the effects on an implantable medical device of atmospheric pressure variations.

The present invention thus relates, according to a first aspect, to an implantable medical device comprising a variable-volume fluid reservoir, the reservoir being deformable under the influence of an atmospheric pressure variation, an inflatable element in fluid connection with the reservoir, an actuator suitable for selectively varying the volume of the fluid reservoir, and a data processing and control unit configured to control a selective variation of the fluid reservoir volume by the actuator, said unit being configured to implement, if an estimate of an atmospheric pressure crosses at least one atmospheric pressure threshold and/or if an estimate of an altitude crosses at least one altitude threshold, at least one of the steps of:
a) controlling, by means of the data processing and control unit, at least one withdrawal of a volume of fluid from the inflatable element to the reservoir by means of the actuator;
b) controlling, by means of the data processing and control unit, at least one injection of a volume of fluid from the reservoir to the inflatable element by means of the actuator.

According to advantageous and non-limiting features, taken alone or in any combination:
the data processing and control unit is configured to:
   implement step a) if the estimate of the atmospheric pressure crosses the at least one atmospheric pressure threshold so that the atmospheric pressure is less than the at least one atmospheric pressure threshold or if the estimate of the altitude crosses the at least one altitude threshold so that the estimate of the altitude is greater than the at least one altitude threshold; and/or to
   implement step b) if the estimate of the atmospheric pressure crosses the at least one atmospheric pressure threshold so that the atmospheric pressure is greater than the at least one atmospheric pressure threshold or if the estimate of the altitude crosses the at least one altitude threshold so that the estimate of the altitude is less than the at least one altitude threshold;
the data processing and control unit is configured to implement, prior to step a) or b), a step a01) or b01) of calculating the volume of fluid to be withdrawn or injected;
the data processing and control unit is configured to implement a step a0) of estimating the atmospheric pressure based on at least one value of fluid pressure in the reservoir;
the data processing and control unit is configured to calculate the volume of fluid to be withdrawn or to be injected depending on an atmospheric pressure range in which the estimate of the atmospheric pressure is newly comprised, a lower limit or an upper limit of the atmospheric pressure range being the atmospheric pressure threshold and/or depending on an altitude range in which the estimate of altitude is newly comprised, a lower limit or an upper limit of the altitude range being the altitude threshold;
the data processing and control unit is configured to calculate the volume of fluid to be withdrawn or to be injected depending on an atmospheric pressure range in which the estimate of the atmospheric pressure is newly comprised, the estimate of the atmospheric pressure being comprised in one of a plurality of atmospheric pressure ranges and/or depending on an altitude range in which the estimate of the altitude is newly comprised, the estimate of the altitude being newly comprised in one of a plurality of altitude ranges;
the data processing and control unit is configured to calculate the volume of fluid to be withdrawn or to be injected depending on an atmospheric pressure range in which the estimate of the atmospheric pressure is newly comprised, the estimate of the atmospheric pressure being newly comprised in one of at least two atmospheric pressure ranges, preferably at least three atmospheric pressure ranges, more preferably at least four atmospheric pressure ranges, and/or depending on an altitude range in which the altitude estimate is newly comprised, the estimate of the altitude being newly comprised in one of at least two altitude ranges, preferably at least three altitude ranges, preferably at least three altitude ranges, more preferably at least four altitude ranges;
the data processing and control unit is configured to calculate the volume of fluid to be withdrawn or to be injected depending on the atmospheric pressure range in which the atmospheric pressure estimate is newly comprised and/or depending on the altitude range in which the estimate of altitude is newly comprised, each atmospheric pressure range and/or each altitude range being associated with a respective fluid volume value, in determining the fluid volume value associated with the atmospheric pressure range in which the estimate of the atmospheric pressure is newly comprised and/or with the altitude range in which the altitude estimate is newly comprised, the volume of fluid to be withdrawn or to be injected corresponding to the determined fluid volume value;
the data processing and control unit is configured to calculate at least one lower limit and/or at least one upper limit of the at least one atmospheric pressure range and/or of the at least one altitude range based on at least one reference atmospheric pressure value and/or at least one reference altitude value and based on at least one atmospheric pressure difference threshold and/or at least one altitude difference threshold;
the data processing and control unit is configured to calculate at least one fluid volume value associated with each atmospheric pressure range and/or with each altitude range, the at least one fluid volume value corresponding to a subdivision of a maximum volume to be withdrawn or to be injected during an atmospheric pressure variation and/or a maximum altitude variation;
the data processing and control unit is configured to implement a step of:
   x) detecting whether the estimate of the atmospheric pressure crosses at least one atmospheric pressure threshold or whether an estimate of an altitude crosses at least one altitude threshold, the at least one atmospheric pressure threshold being a lower or upper limit of an atmospheric pressure range and the at least one altitude threshold being a lower or upper limit of an altitude range;
the data processing and control unit is configured to exchange data with an external control element comprising a barometer and/or an altimeter and/or a GPS, the data processing and control unit being configured to estimate the atmospheric pressure based on atmospheric pressure measurement data from the barometer and/or to estimate the altitude based on altitude measurement data from the altimeter and/or from the GPS;
the data processing and control unit is configured to record the atmospheric pressure measurement by the barometer as an estimate of the atmospheric pressure if the atmospheric pressure measured is comprised between 1054 mbar and 694 mbar and/or to record the altitude measurement by the altimeter as an estimate of the altitude if the measured altitude is comprised between 0 meters and 3000 meters above sea level;
the data processing and control unit is configured to calculate the volume of fluid to be withdrawn depending on an estimate of a second fluid volume which was injected into the inflatable element due to a deformation of the reservoir caused by a reduction of the atmospheric pressure and/or to an increase of the altitude;
the data processing and control unit is configured to calculate the volume of fluid to be injected depending on an estimate of a second fluid volume which was withdrawn from the inflatable element due to a deformation of the reservoir caused by an increase of the atmospheric pressure and/or a decrease of the altitude;
the data processing and control unit is configured to exchange data with an external control element comprising a barometer and/or an altimeter and/or a GPS and configured, if a command is implemented by a patient in whom the device is implanted via the external control element, to record an atmospheric pressure measurement by the barometer and/or an altitude measurement by the altimeter and/or a measurement of the altitude by the GPS;
the data processing and control unit is configured to update the lower limit and the upper limit of the atmospheric pressure range(s) due to the recording of an atmospheric pressure measurement by the barometer and/or of an altitude measurement from the altimeter and/or an altitude measurement from the GPS so that an atmospheric pressure range is centered on the atmospheric pressure measurement, and/or update the lower limit and the upper limit of the altitude range(s) so that an altitude range is centered on the altitude measurement;
the data processing and control unit is configured to increase, preferably to double, the extent of the atmospheric pressure range centered on the atmospheric pressure measurement and/or of the altitude range centered on the altitude measurement;
the data processing and control unit is configured to calculate a volume of fluid to be withdrawn from the inflatable element to the reservoir by means of the actuator or to inject from the reservoir to the inflatable element by means of the actuator by applying the atmospheric pressure or altitude measurement to a function, due to the recording of an atmospheric pressure measurement by the barometer and/or of an altitude measurement by the altimeter and/or an altitude measurement by the GPS;
the data processing and control unit is configured to exchange data with an external control element comprising a barometer and/or an altimeter for recording, at a predetermined frequency, an atmospheric pressure measurement by the barometer and/or an altitude measurement by the altimeter;
the data processing and control unit is configured to control an increase or a decrease in the volume of the reservoir by the actuator so as to allow the withdrawal or the injection of the volume of fluid from the inflatable element to the reservoir;
the data processing and control unit is configured to implement step a) periodically and/or step b) periodically;
the device is configured to be implanted into a human or animal body to selectively block an anatomical passage of said human or animal body selected from at least one of the following passages: a urethra, a gastric passage, a colon or a rectum, or comprising an elongate shaped inflatable element configured to be used as penile implants.

According to a second aspect, the invention relates to an assembly comprising an implantable medical device presented previously and an external control element suitable for exchanging data with the implantable medical device and configured to be used by an individual into whom the medical device is implanted, in which the implantable medical device and the external control element comprise communication means suitable for communicating with one another.

According to a third aspect, the invention relates to a method for compensating an atmospheric pressure variation to which an implantable medical device is exposed, said medical device comprising a variable-volume fluid reservoir able to deform under the influence of an atmospheric pressure variation, an inflatable element in fluid connection with the reservoir, an actuator suitable for selectively varying the volume of the fluid reservoir, and a data processing and control unit configured to control a selective variation of the volume of the fluid reservoir by the actuator, the method comprising at least one of the steps implemented by the data processing and control unit, if an estimate of an atmospheric pressure crosses at least one atmospheric pressure threshold or if an estimate of an altitude crosses at least one altitude threshold, of:
a) sending by the control unit of at least one order to the actuator to vary the volume of the reservoir for withdrawing a volume of fluid from the inflatable element to the reservoir;
b) sending by the control unit of at least one order to the actuator to vary the volume of the reservoir for injecting a volume of fluid from the reservoir to the inflatable element.

According to a fourth aspect, the invention relates to a computer program product comprising code instructions for the execution of a compensation method presented previously, when said program is executed by a computer.

According to a fifth aspect, the invention relates to a storage means readable by computing equipment on which a computer program product comprises code instructions for the execution of a compensation method presented previously.

### DESCRIPTION OF THE FIGURES

Other features and advantages of the present invention will appear upon reading the description that follows of a preferred embodiment. This description will be given with reference to the appended figures, in which:

FIG. 1 illustrates an overall view of a medical device that is implantable in the body of an individual, and of a control element outside the body of the individual;

FIG. 2 is a schematic section view of the interior of the housing according to one embodiment;

FIG. 3 shows a breakdown of atmospheric pressure ranges;

FIG. 4 shows a breakdown of atmospheric pressure ranges according to another embodiment;

FIG. 5 shows the steps in the method for compensating a variation of atmospheric pressure to which an implantable medical device is exposed.

### DETAILED DESCRIPTION OF THE INVENTION

### Device

According to a first aspect, a medical device that is implantable into an individual is proposed. What is meant in the present text by “individual” is a human being or an animal. The device is an implantable active medical device capable of blocking a natural passage such as a urethra (in a man), the bladder neck (in a woman), a gastric passage, a colon or even a rectum. In one case of application to a urethra or to a bladder neck, the device allows in particular combatting urinary incontinence by means of an artificial sphincter capable of blocking the urethra or the bladder neck. However, the proposed device is more generally a device comprising a fluid circuit sensitive to pressure variations particularly generated by variations of altitude. Among the other forms that the device can take can be cited in particular penile implants and gastric constriction bands.

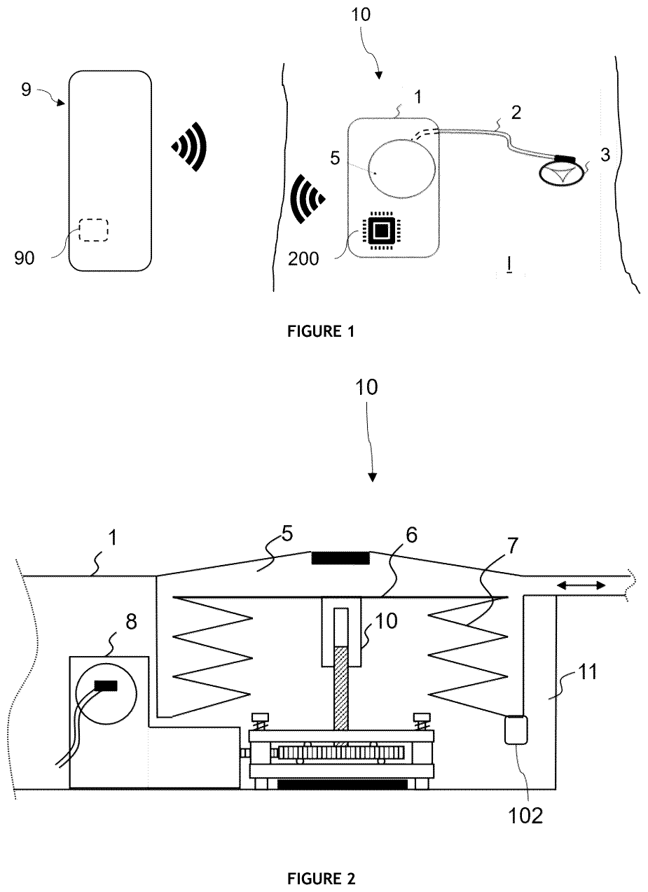

A medical device that is implantable in a human or animal body is illustrated by way of a non-limiting example in FIGS. 1 and 2.

The implantable device 10 comprises:
a sealed housing 1 containing a gas,
an inflatable element 3 suitable for being implanted into the body of the individual outside the housing,
a fluid circuit comprising a fluid reservoir 5 having a variable fluid volume arranged in the housing and a fluid connection 2 between said reservoir 5 and said inflatable element 3,
an actuator 8 arranged in the housing 1 and mechanically coupled to a portion of the fluid reservoir 5 to selectively vary the fluid volume in said reservoir,
a data processing and control unit 200 configured to control a selective variation of the volume of the fluid reservoir 5 by the actuator 8 and therefore configured to control the actuator so as to transfer fluid between the reservoir 5 and the inflatable element 3. The unit 200 is also configured to implement a method for compensating an atmospheric pressure variation to which the implantable medical device is exposed.

### Fluid Circuit

The fluid circuit is suitable for being filled with a fluid, particularly a liquid. A variation of the volume of the reservoir 5 causes a variation in the pressure in the fluid circuit. More particularly, a reduction in the volume of the reservoir 5 causes a transfer of fluid from the reservoir 5 to the inflatable element 3, and causes an increase in the pressure in the fluid circuit. Conversely, an increase in the volume of the reservoir 5 causes a transfer of fluid from the inflatable element 3 to the reservoir 5, and causes a reduction of the pressure in the fluid circuit.

The reservoir 5 is preferably a variable-volume fluid reservoir suitable for being deformed under the influence of an atmospheric pressure variation. The reservoir 5 can therefore comprise an elastically deformable portion which deforms depending on atmospheric pressure variations. This deformation can cause a variation in the volume of the reservoir 5 and therefore transfers of fluid between the reservoir 5 and the inflatable element 3.

The reservoir 5 also comprises an opening allowing transferring the fluid from and outside the reservoir 5 to the inflatable element 3 via the fluid connection 2.

The fluid connection 2 can consist of a tube 2 arranged between the reservoir 5 and the inflatable element 3. A first end of the tube 2 opens into the reservoir 5, and a second end of the tube 2 opens into the inflatable element 3.

The inflatable element 3 can be an inflatable occlusive cuff, in particular when the device 10 is an artificial urinary sphincter. The inflatable occlusive cuff 3 filled with fluid can be suitable for completely or partially surrounding the passage to be blocked.

As a variant, the inflatable element 3 can be an inflatable penile implant, and thus have an elongate shape, in particular when the device 10 is an erectile prosthesis.

### Housing

The housing 1, the fluid connection 2 and the inflatable element 3 are suitable for being implanted into the body of an individual I, the contours of which are shown schematically in FIG. 1 on either side of this assembly.

The housing 1, in particular the interior volume 11 of the housing 1 surrounding the reservoir 5, contains a gas, for example an inert gas.

### Sensor

Advantageously, the housing 1 encloses a reservoir sensor 102 suitable for measuring a representative value of the fluid pressure in the reservoir 5. The reservoir sensor 102 can for example be a force sensor or a pressure sensor.

### External Control Element

In a particularly advantageous manner, an external control element 9, such as a remote control, outside the body of the patient, is usable by the patient or a third party to communicate wirelessly with the medical device 10.

In certain embodiments, a barometer 90, i.e. an atmospheric pressure sensor, is comprised in the device 10, for example in the housing 1. According to another embodiment, the barometer 10 is arranged on an outside wall of the housing 1 and is configured to communicate with the device 10. According to still another embodiment, the barometer 90 can be arranged in the external control element 9 outside the body of the individual in which the device 10 is implanted. The barometer 90 is suitable for measuring a representative value of an atmospheric pressure to which the implantable medical device 10 is subjected. In the case of a barometer 90 arranged in the external control element 9, a measurement of the representative value of the atmospheric pressure can thus be carried out via the external control element 9, for example when the patient himself activates a command of the external control element 9. Also in certain embodiments, an altimeter 92, i.e. an altitude sensor, can be arranged in the external control element 9 outside the body of the individual in which the device 10 is implanted. The altimeter 92 is adapted to measure a representative value of an altitude at which the implantable medical device is located. A measurement of the representative altitude value can also be carried out when a user controls the device 10 via the external control element 9.

The barometer 90 and the altimeter 92 or a GPS (“Global Positioning System”) can also be configured to implement a measurement of atmospheric pressure or an altitude measurement, respectively, at a predetermined frequency.

### Actuator

The actuator 8 is suitable for controlling a variation in the volume of the reservoir 5. In a certain embodiment, the actuator 8 is suitable for controlling a linear movement of the movable wall 6, the bellows 7 being suitable for extending or compressing depending on said linear movement of the movable wall 6 commanded by the actuator 8.

The actuator 8 can be selected from any electromechanical system allowing transforming electrical energy into mechanical movement with the power required to allow the movement at a force and speed required by the movable wall 6 of the variable-volume reservoir 5. The actuator 8 can in particular be a piezo-electric actuator, an electromagnetic actuator which can comprise an electromagnetic motor with or without brushes, coupled or not to a reduction gear, an electroactive polymer or a shape-memory alloy.

### Data Processing and Control Unit

The device 10 comprises a data processing and control unit 200 configured to control the actuator 8 so as to move the movable wall 6 of the reservoir 5 to a position corresponding to the determined volume. More particularly, in the example illustrated in FIG. 2, the control unit 200 is configured to transmit an operating order to a motor of the actuator 8 in one direction or in the other depending on whether an increase or a reduction in the volume of the reservoir 5 is required.

As previously explained, an atmospheric pressure or altitude variation can cause deformations of the reservoir 5 of the device 10. These deformations can cause an injection of fluid into the inflatable element 3 or an uncontrolled withdrawal of fluid from the inflatable element 3 and therefore an increase or a reduction of the fluid pressure in the inflatable element 3. Yet, if the fluid pressure in the inflatable element 3 is too high, this can cause degradations of the tissues of the anatomical passages that the inflatable element 3 surrounds. Conversely, if the fluid pressure in the inflatable element 3 is too high, the anatomical passage surrounded by the inflatable element 3 is able to not be sufficiently blocked which can cause, in the example case where the passage is a urethra, an incontinence episode.

For example, in the embodiment illustrated in FIG. 2, the bellows 7 forms a part of the wall of the variable-volume reservoir. The bellows is formed from a plurality of convolutions which can be elastically deformable. The bellows 7 can be a in the form of an accordion bellows, the convolutions corresponding to the folds of the accordion. Statically, i.e. when no stress is exerted by the actuator 8 to vary the volume of the reservoir, an atmospheric pressure variation can cause a deformation of the convolutions, thus varying the volume of the reservoir.

It is therefore necessary to be capable of compensating this injection or this withdrawal by withdrawing or by injecting, respectively, fluid into or from the inflatable element 3.

The unit 200 is configured to control an injection or a withdrawal of a fluid volume to be compensated. For this purpose, the unit 200 is configured to determine whether an atmospheric pressure is less than or greater than at least one atmospheric pressure threshold and/or to determine whether an altitude is less than or greater than at least one altitude threshold. This determination allows the triggering or not of an injection or a withdrawal by the unit 200.

It is understood that the unit is configured to react to atmospheric pressure and/or altitude variation. What is meant by atmospheric pressure is the atmospheric pressure to which the device 10 is exposed. What is meant by altitude is the altitude at which the device 10 is located. These quantities are linked in that the atmospheric pressure varies linearly as a function of altitude. During an increase in altitude, the atmospheric pressure decreases and conversely, during a reduction in altitude, the atmospheric pressure increases.

For the sake of clarity, the continuation of the description will be divided into two parts. The first part relates to the configuration of the unit 200 to compensate as regards atmospheric pressure values. The second part relates to the configuration of the unit 200 to compensate as regards altitude values. Obviously, the atmospheric pressure and the altitude being inversely linked (when one increases, the other decreases), the embodiment relating to altitude is the same as the embodiment, reversed.

### Atmospheric Pressure Embodiment

The unit 200 is advantageously configured to estimate an atmospheric pressure value.

The atmospheric pressure value can be measured by the barometer 90, then communicated to the unit 200. According to a certain embodiment, the unit 200 is configured to record an atmospheric pressure value measured by the barometer 90 due to a command by an individual via the external control element 9. According to a certain embodiment, the unit 200 is configured to record an atmospheric pressure value measured by the barometer 90 at a predetermined frequency.

An estimate of the value of atmospheric pressure is thus obtained. This estimate corresponds to the atmospheric pressure to which the device is exposed at the instant tn.

According to another embodiment, the unit 200 is configured to implement a step a0) of estimating the atmospheric pressure based on at least one value of fluid pressure in the reservoir 5. The fluid pressure value in the reservoir can be measured by the reservoir sensor 102. The manner of estimating the atmospheric pressure depending on a fluid pressure value in the reservoir 5 will not be detailed in this description.

The unit 200 is also configured to store a reference atmospheric pressure value P_atmₜ₀. The reference value can typically be acquired by the barometer. The reference value was recorded by the unit 200 at an instant t0 corresponding to the activation of the device 10. The reference value is therefore fixed at the activation of the device 10 and is not destined to be modified (though reconfigurable in case of necessity).

The unit 200 is configured to calculate at least one lower limit and/or at least one upper limit of at least one atmospheric pressure range based on at least one reference atmospheric pressure value and based on at least one atmospheric pressure difference threshold. In fact, the unit 200 is configured to determine the atmospheric pressure ranges. Briefly, each range is associated with a volume of fluid to be injected or to be withdrawn and, depending on an estimate which will be made of an atmospheric pressure, the unit 200 is configured to determine in which pressure range the estimate is newly comprised and thus determine a volume of fluid to be injected into the inflatable element 3 from the reservoir 5 or to be withdrawn from the inflatable element 3. In this manner, the unit 200 is configured to compensate, within the device, the deformations of the reservoir 5 caused by variations of atmospheric pressure.

The unit 200 is configured to determine at least one range, preferably several. In fact, preferably, the unit 200 is configured to determine at least three ranges, more preferably at least four ranges.

As explained, each atmospheric pressure range is determined based on at least one reference atmospheric pressure value and based on at least one atmospheric pressure difference threshold. The reference atmospheric pressure value corresponds ideally to a maximum atmospheric pressure value, corresponding for example to atmospheric pressure at an altitude of 0 meters relative to sea level, or approximately 1054 mbar. Moreover, the atmospheric pressure difference threshold corresponds to an extent of the predetermined range. Typically, this difference threshold is configured upon the activation of the device 10 and is not destined to be modified (although reconfigurable in case of necessity).

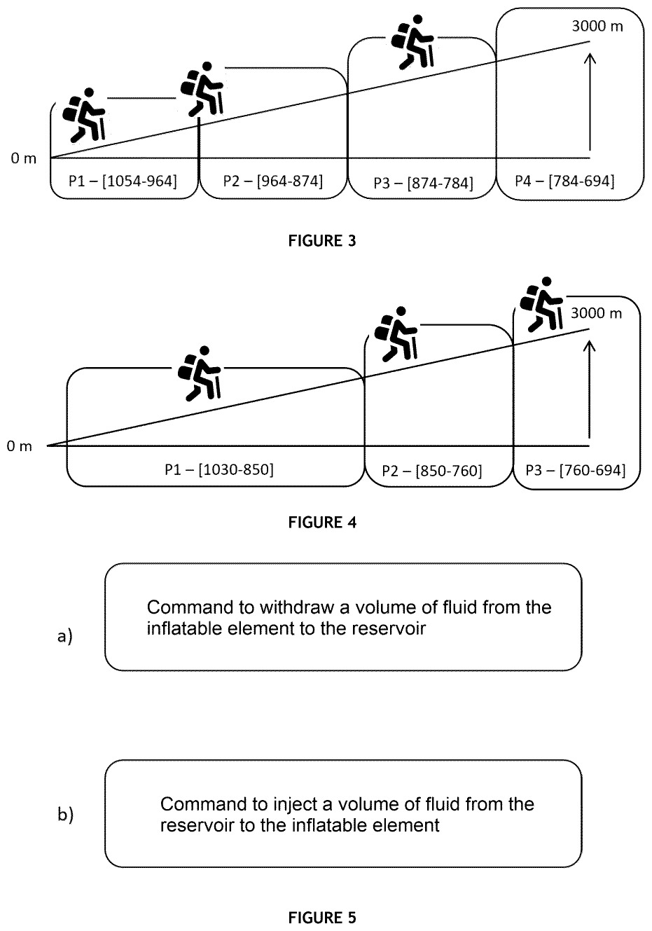

Thus, with reference to FIG. 3, if a first difference threshold is fixed at 90 mbar, a first range P1 will extend over 90 mbar from 1054 mbar to 964 mbar. The upper limit of P1 is 1054 mbar and the lower limit of P1 is 964 mbar. If, for all the ranges determined by the unit 200, the difference threshold is identical, then the upper limit of a second range P2 is 964 mbar and the lower limit of P2 is 874 mbar. Identically, a third range P3 has as its upper limit 874 mbar and as a lower limit 784 mbar. A fourth range P4 has as its upper limit 784 mbar and as a lower limit 694 mbar. According to another embodiment, the atmospheric pressure thresholds (i.e. the extents of the ranges) are not identical from one range to another.

In the example presented above, it is noticed that the upper limit of P1 is equal to 1054 mbar and the lower limit of P4 is equal to 694 mbar. This corresponds, in altitude, to an altitude range extending from 0 meter (1054 mbar) to 3000 meters (694 mbar) above sea level.

In fact, the device 10 is preferably configured to be used at certain atmospheric pressures and/or altitudes. The unit 200 can therefore be configured to compensate deformations of the reservoir 5 caused by variations of atmospheric pressure and/or altitude for atmospheric pressures or altitudes respectively comprised between 1054 mbar and 694 mbar or 0 meter and 3000 meters above sea level. However the unit 200 can be configured to compensate deformations of the reservoir 5 caused by variations in atmospheric pressure and/or of altitudes for different atmospheric pressures or altitudes (for example for altitudes comprised between 0 meter and 4000 meters above sea level).

The unit 200 is configured to associate with each range a value of fluid volume. This value of fluid volume corresponds to a volume of fluid to be injected into the inflatable element 3 or to withdraw from the inflatable element 3 if an estimate of atmospheric pressure is newly comprised in a certain range. The volume of fluid associated with each range is ideally configured upon the activation of the device 10 and is not destined to be modified (though reconfigurable in case of necessity). Advantageously, the unit 200 is configured to calculate values of fluid volume associated with each range based on a maximum fluid volume corresponding to the fluid volume to be withdrawn or to be injected during a maximum atmospheric pressure variation. Thus, each value of fluid volume associated with the ranges corresponds to a subdivision of the maximum fluid volume.

The maximum volume of fluid is typically an estimate of a volume which is injectable into the inflatable element 3 from the reservoir 5 in the case of a maximum reduction in atmospheric pressure (for example a reduction of atmospheric pressure of 360 mbar corresponding to an increase in altitude of 3000 meters). This maximum volume of fluid thus corresponds, reciprocally, with an estimate of a volume which is able to be withdrawn from the inflatable element 3 to the reservoir 5 in the case of a maximum increase of atmospheric pressure (for example an increase in atmospheric pressure of 360 mbar corresponding to a reduction of altitude of 3000 meters). The values of fluid volumes associated with the ranges can be equal to a range of another, or different.

According to a particular embodiment, the unit 200 is configured to update the ranges. In other words, the ranges are dynamic. More precisely, the unit 200 is configured to update the ranges when an atmospheric pressure value is measured by the barometer 90 (for example due to a command by an individual via the external control element 9). What is meant by “update” is that the lower limit and the upper limit of the ranges are modified. The limits are modified depending on the atmospheric pressure value measured by the barometer 90. Preferably, the ranges are modified so that a range, preferably the first range P1, is re-centered on the atmospheric pressure value measured by the barometer 90. For example, if the atmospheric pressure value measured by the barometer 90 is 940 mbar (value comprised in the range P2 according to the example of FIG. 3), the range P1 will be modified so that its lower limit corresponds to 895 mbar and its upper limit corresponds to 985 mbar, the value of 940 mbar being central for this new range P1. In this example, it is understood that the example of ranges extending over 90 mbar has been repeated. The range P2 will have an upper limit of 895 mbar and a lower limit of 815 mbar (895 mbar-90 mbar). Similarly, the range P3 will have an upper limit of 815 mbar and a lower limit of 725 mbar (815 mbar-90 mbar). The range P4 will have an upper limit of 725 mbar and preferably a lower limit of 694 mbar because, as previously explained, the unit 200 is preferable configured to be used at an atmospheric pressure greater than or equal to 694 mbar. The advantages procured by the embodiments with dynamic ranges will be detailed later, in light of other elements of the description.

According to a particular embodiment, the unit 200 is configured to modify the extent of the range which is re-centered on the atmospheric pressure value measured by the barometer 90. Preferably, this extent is increased and is, still preferably, doubled relative to the initial extent. In this manner, starting from the center of the newly calculated range, a variation of more than 90 mbar, in one direction or the other, will induce a single range change (for example based on the center of the range P2, a variation of more than 90 mbar will only induce a change from the range P2 to the range P3 or from the range P2 to the range P1). This allows avoiding the withdrawal or the injection of fluid from or to the inflatable 3 which is too sudden. In other words, this allows a more gradual and more precise compensation of the atmospheric pressure variation. If the example of the previous paragraph is repeated, the unit 200 is configured to re-center the range P1 on 940 mbar and to increase the extent of the range P1 to 180 mbar (90 mbar multiplied by two) as illustrated in FIG. 4. Thus the range P1 will have as an upper limit 1030 mbar (940 mbar+90 mbar) and as lower limit 850 mbar (940 mbar−90 mbar). On the other hand, the extent of the other ranges will remain unchanged. Thus, the range P2 will have as its upper limit 850 mbar and as lower limit 760 mbar (850 mbar−90 mbar).

The unit 200 is preferably configured to implement a step x) of detecting whether the estimate of the atmospheric pressure crosses at least one atmospheric pressure threshold and this threshold is preferably a lower or upper limit of an atmospheric pressure range. In other words, the unit 200 is preferably configured to implement a step of detecting a change of the range in which the estimate of the atmospheric pressure is newly comprised. What is meant by “newly” is thus that the estimate of the atmospheric pressure was previously comprised in another range and that the crossing of the atmospheric pressure threshold brought it into a new range. It is recalled that the estimate of the atmospheric pressure estimate is estimated at an instant tn. A change of range signifies that the estimate of the atmospheric pressure value has crossed an atmospheric pressure threshold (corresponding to a limit of a range). This also signifies that, at the instant tn, the individual in which the device 10 is implanted is exposed to an atmospheric pressure lower or upper than at a preceding instant (possibly linked to variations of altitude).

To detect a change of the range in which the estimate of the atmospheric pressure is newly comprised, the unit 200 is advantageously configured to determine whether the difference between the estimate of the atmospheric pressure and the reference atmospheric pressure value is greater than an atmospheric pressure difference threshold. For this purpose, the unit 200 is configured to determine a difference between an estimate of the atmospheric pressure and the reference atmospheric pressure value. In other words, the unit 200 is capable of evaluating the difference between an estimate and the reference value, the reference value having been recorded before the estimate. It is understood here that the unit 200 is configured to detect an increase or a decrease of atmospheric pressure beyond a certain atmospheric pressure increase or decrease threshold. For example, let us assume that the atmospheric pressure estimate is 900 mbar and that the reference atmospheric pressure is 1054 mbar. A difference between the atmospheric pressure estimate and the reference atmospheric pressure value is 154 mbar. The reference value being acquired prior to the estimate, it is understood that the device 10 is subjected to a reduction in atmospheric pressure of 154 mbar (which can correspond to an increase of altitude).

Then this difference is compared to an atmospheric pressure difference threshold or to a set of atmospheric pressure difference thresholds. As previously explained, an atmospheric pressure difference threshold corresponds to the extent of a range. Taking the case where the ranges have equal extents, and therefore the atmospheric pressure difference thresholds are equal from one range to another and equal to 90 mbar. In the example presented above, the atmospheric pressure difference is equal to 154 mbar, which is greater than 90 mbar but less than 180 mbar (or 2×90 mbar). The pressure difference is therefore greater than a difference threshold but not to a set of two difference thresholds. The unit is configured to deduce from this that the range in which the atmospheric pressure estimates (i.e. 900 mbar) is the range P2. Depending on this, the unit 200 is configured to determine whether this range is identical or not to the range in which the atmospheric pressure estimate was comprised at an instant tn−1 earlier than the instant tn. If, at the instant tn−1, the atmospheric pressure estimate was comprised in the range P1, then the unit 200 is configured to detect a change of range and to warn that the new atmospheric pressure estimate (at the instant tn) is less than an atmospheric pressure threshold, namely the lower limit of the range P1. The unit 200 is thus configured, if a change of ranges in which the atmospheric pressure estimate is comprised is detected, to determine whether the atmospheric pressure estimate is less than or greater than an atmospheric pressure threshold.

If it is determined that the estimate of the atmospheric pressure has crossed an atmospheric pressure threshold (in such a manner that the estimate of the atmospheric pressure at the instant tn is comprised in a range different from the atmospheric pressure estimate at the instant tn−1), the unit 200 is configured to control a withdrawal or an injection of fluid. In fact, the range changing phenomenon indicates that the reservoir 55 is probably deformed to as to inject or withdraw fluid in the inflatable element without this being desired. These undesired injections or withdrawals can affect the integrity of the tissues surrounded by the inflatable element (in the event of an overpressure caused by an injection of fluid), or on the contrary urine leaks (in the event of insufficient pressure caused by a withdrawal of fluid).

Consequently, the unit 200 is preferably configured to implement a step a01) or b01) of calculating a volume of fluid to be withdrawn or to be injected. Advantageously, the unit 200 is configured to select the volume of fluid associated with the range in which the atmospheric pressure estimate is newly comprised, given that preferably, as explained earlier, a volume of fluid is associated with each range.

The unit 200 is configured to implement, if the estimate of the atmospheric pressure crosses the at least one atmospheric pressure threshold so that the atmospheric pressure is less than the at least one atmospheric pressure threshold, a step a) of commanding at least one withdrawal of the fluid volume from the inflatable element 3 to the reservoir 5 by means of the actuator 8. To implement this, the unit is configured to control an increase of the volume of the reservoir 5 which allows the withdrawal of fluid in the inflatable element 3. In this particular case, the unit 200 compensates a reduction of atmospheric pressure (possibly linked to an increase in altitude) [by] causing an injection of fluid into the inflatable element 3 from the reservoir 5. This particular case corresponds to the example presented above corresponding to a change of range from P1 to P2. The volume of fluid to be withdrawn can be withdrawn at one time or at several times.

In addition, the unit 200 is configured to implement, if the estimate of the atmospheric pressure crosses the at least one atmospheric pressure threshold so that the atmospheric pressure is greater than the at least one atmospheric pressure threshold, a step b) of commanding at least one injection of a volume of fluid from the reservoir 5 to the inflatable element 3. To implement this, the unit is configured to command a reduction of the volume of the reservoir 5 which allows the injection of fluid into the inflatable element 3. In this particular case, the unit 200 compensates an increase in atmospheric pressure (possibly linked to an altitude reduction) [by] causing a withdrawal of fluid in the inflatable element 3.

Moreover, the embodiments with dynamic ranges previously detailed allow avoiding the situation that will be described. The unit 200 estimates an atmospheric pressure value comprised in the range P2, near the upper limit of the range P2, so that this limit is crossed, which results in commanding a fluid withdrawal. Then, due to a command by an individual via the external control element 9, the barometer 90 measures an atmospheric pressure value which updates the estimated atmospheric pressure value (and corrects it because the value measured by the barometer is assumed to be more reliable than the estimate by the unit 200). This measured value is comprised in the range P1, near the lower limit of P1 (and therefore near the upper limit of P2), which results in commanding a fluid injection. If the patient remains at the same altitude, alternating passages from one range to the other risk occurring with repeated injections and withdrawals of fluid, which induces a useless consumption of energy by the device 1 and possible discomfort for the patient. The embodiments with dynamic ranges allow avoiding this phenomenon, which occur when the patient remains at an atmospheric pressure/an altitude near the value of a limit of a range, by adapting the limits of ranges.

Advantageously, the unit 200 is configured to command an injection or a withdrawal of a volume of fluid when an atmospheric pressure value is measured by the barometer 90 (in particular due to a command by an individual via the external control element 9). In other words, instead of injections of withdrawals of fluid controlled automatically by the unit 200 depending on an estimate of the atmospheric pressure, injections or withdrawals of fluid can be controlled by a user via the external control element 9. More precisely, the unit 200 is configured to calculate a volume of fluid to be injected or to be withdrawn due to a command of an individual via the external control element 9 (and therefore due to the measurement of an atmospheric pressure value by the barometer 90). Preferably, this volume is calculated based on a function and on the value of the atmospheric pressure measured by the barometer 90. Preferably, this function is an affine function, i.e. of the type y32 ax+b, x corresponding to the atmospheric pressure value measured by the barometer 90, y corresponding to the volume of fluid to be withdrawn or injected, a and b being real constants. It is therefore understood that the unit 200 is configured to calculate a volume of fluid to be withdrawn or injected by applying the volume to the atmospheric pressure value measured by the barometer 90. It is appropriate to note that, if the atmospheric pressure value measured by the barometer 90 was identical to the estimate of the current atmospheric pressure (what is meant by “current” is the last estimate of the current atmospheric pressure determined by the unit 200), the volume of fluid to be withdrawn or injected would be zero. In addition, it is understood that the unit 200 is configured to determine whether the measured atmospheric pressure value is greater than or less than the estimate of the current atmospheric measurement so that the unit 200 is configured to determine whether a certain volume of fluid is to be injected or withdrawn. This embodiment is combined with the embodiment involving dynamic ranges so as to ensure consistent compensation of atmospheric pressure variation. According to this embodiment, the unit 200 therefore allows more regular compensation of possible atmospheric pressure variations, guaranteeing better comfort for the patient and greater safety. In addition, according to this embodiment, compensation can be directly controlled, commanded by the patient.

Consequently, the device 10 is suitable for compensating atmospheric pressure variations. The device 10 is therefore advantageously suitable for compensating deformations of the reservoir 5 caused by atmospheric pressure variations. The individual into whom the device 10 is implanted can expose himself to atmospheric pressure variations (for example increasing or decreasing altitude) without risking degrading tissues surrounding his anatomical passage. Preferably, the unit 200 is therefore configured to implement at least steps a) and b) periodically, for example once per hour, more preferably once every half-hour, more preferably every 10 minutes.

### Altitude Embodiment

The unit 200 is advantageously configured to estimate and altitude value. The altitude value can be measured by the altimeter 92 of the external control element 9, then communicated to the unit 200. According to a certain embodiment, the unit 200 is configured to record an altitude value measured by the altimeter 92 due to a command of an individual via the external control element 9. According to a certain embodiment, the unit 200 is configured to record an altitude value measured by the altimeter 92 at a predetermined frequency.

The unit 200 is also configured to store a reference altitude value A_atmₜ₀. The reference value can typically be acquired by the altimeter. The reference value was recorded by the unit 200 at an instant t0 corresponding to the activation of the device 10. The reference value is therefore fixed upon the activation of the device 10 and is not destined to be modified (though reconfigurable in case of necessity).

The continuation of the embodiment regarding altitude is similar to the embodiment regarding atmospheric pressure, within a few differences. First of all, it is understood that the estimate of atmospheric pressure, the atmospheric pressure thresholds, the atmospheric pressure ranges, etc. correspond respectively in the embodiment regarding altitude to an estimate of altitude, altitude thresholds, altitude ranges, etc.

In addition, as explained previously, a reduction in altitude involves an increase in atmospheric pressure and, conversely, an increase in altitude involves a reduction in atmospheric pressure. It is therefore understood that a reduction in altitude will be compensated by an injection of fluid into the inflatable element while an increase in altitude will be compensated by a withdrawal of fluid from the inflatable element.

Thus, the embodiment regarding altitude can simply be based on the embodiment regarding atmospheric pressure by taking these differences into account.

### Assembly

According to a second aspect, an assembly comprising an implantable medical device 10 is proposed, as described above, and an external control element 9 suitable for being used by an individual, for example by an individual in whom the system is implanted. The implantable medical device 10 and the external control element 9 comprise communication means suitable for communicating with one another. The communication means of the implantable device 10 can be integrated into the housing 1.

### Method

Referring to FIG. 5, according to a third aspect, a method for compensating an atmospheric pressure variation to which the implantable medical device 10 is exposed is proposed, comprising at least one of the steps implemented by the data processing and control unit, if an estimate of an atmospheric pressure crosses at least one atmospheric pressure threshold or if an estimate of an altitude crosses at least one altitude threshold, of:
a) sending by the control unit of at least one order to the actuator to vary the volume of the reservoir to withdraw a volume of fluid from the inflatable element to the reservoir;
b) sending by the control unit of at least one order to the actuator to vary the volume of the reservoir to inject a volume of fluid from the reservoir to the inflatable element.

### Program Product and Storage Means

According to a fourth aspect, a computer program product is proposed comprising code instructions for the execution off the method for compensating a variation of atmospheric pressure to which the implantable medical device 10 is exposed, when said program is executed by a computer.

According to a sixth aspect, a storage means readable by computing equipment is proposed, in which a computer program product comprises code instructions for the execution of the method [for compensating] a variation of atmospheric pressure to which the implantable medical device 10 is exposed.

The invention is not limited to the embodiment described and shown in the appended figures. Modifications remain possible, in particular from the point of view of the constitution of various technical features or by substitution of technical equivalents, without however departing from the general teaching.

1. An implantable medical device comprising a variable-volume fluid reservoir, the reservoir being deformable under the influence of an atmospheric pressure variation, an inflatable element in fluid connection with the reservoir, an actuator suitable for selectively varying the volume of the fluid reservoir, and a data processing and control unit configured to control a selective variation of the volume of the fluid reservoir by the actuator, the data processing and control unit being configured to implement, if an estimate of an atmospheric pressure crosses at least one atmospheric pressure threshold and/or if an estimate of an altitude crosses at least one altitude threshold, at least one of the steps of:
a) controlling, by means of the data processing and control unit, at least one withdrawal of a volume of fluid from the inflatable element to the reservoir by means of the actuator;
b) controlling, by means of the data processing and control unit, at least one injection of a volume of fluid from the reservoir to the inflatable element by means of the actuator.

2. The device according to claim 1, wherein the data processing and control unit is configured to:
implement step a) if the estimate of the atmospheric pressure crosses the at least one atmospheric pressure threshold so that the atmospheric pressure is less than the at least one atmospheric pressure threshold or if the estimate of the altitude crosses the at least one altitude threshold so that the estimate of the altitude is greater than the at least one altitude threshold; and/or to
implement step b) if the estimate of the atmospheric pressure crosses the at least one atmospheric pressure threshold so that the atmospheric pressure is greater than the at least one atmospheric pressure threshold or if the estimate of the altitude crosses the at least one altitude threshold so that the estimate of the altitude is less than the at least one altitude threshold.

3. The device according to claim 1, wherein the data processing and control unit is configured to implement, prior to step a) or b), a step a01) or b01) of calculating the volume of fluid to be withdrawn or injected.

4. The device according to claim 1, wherein the data processing and control unit is configured to implement a step a0) of estimating the atmospheric pressure based on at least one value of fluid pressure in the reservoir.

5. The device according to claim 1, wherein the data processing and control unit is configured to calculate the volume of fluid to be withdrawn or to be injected depending on an atmospheric pressure range wherein the atmospheric pressure estimate is newly comprised, a lower limit or an upper limit of the atmospheric pressure range being the atmospheric pressure threshold, and/or depending on an altitude range wherein the altitude estimate is newly comprised, a lower limit or an upper limit of the altitude range being the altitude threshold.

6. The device according to claim 1, wherein the data processing and control unit is configured to calculate the volume of fluid to be withdrawn or to be injected depending on an atmospheric pressure range wherein the atmospheric pressure estimate is newly comprised, the estimate of the atmospheric pressure being comprised in one of a plurality of atmospheric pressure ranges, and/or depending on an altitude range wherein the altitude estimate is newly comprised, the estimate of the altitude being newly comprised in one of a plurality of ranges of altitude.

7. The device according to claim 6, wherein the data processing and control unit is configured to calculate the volume of fluid to be withdrawn or to be injected depending on an atmospheric pressure range wherein the atmospheric pressure estimate is newly comprised, the estimate of the atmospheric pressure being newly comprised in one of at least two atmospheric pressure ranges and/or depending on an altitude range wherein the altitude estimate is newly comprised, the estimate of the altitude being newly comprised in one of at least two altitude ranges.

8. The device according to claim 6, wherein the data processing and control unit is configured to calculate the volume of fluid to be withdrawn or to be injected depending on the atmospheric pressure range wherein the atmospheric pressure estimate is newly comprised, and/or depending on the altitude range wherein the altitude estimate is newly comprised, each atmospheric pressure range and/or each altitude range being associated with a respective fluid volume value, in determining the fluid volume value associated with the atmospheric pressure range wherein the atmospheric pressure estimate is newly comprised and/or with the altitude range wherein the altitude estimate is newly comprised, the volume of fluid to be withdrawn or to be injected corresponding to the determined fluid volume value.

9. The device according to claim 5, wherein the data processing and control unit is configured to calculate at least one lower limit and/or at least one upper limit of the at least one atmospheric pressure range and/or of the at least one altitude range based on the at least one reference atmospheric pressure value and/or at least one reference altitude value and based on at least one atmospheric pressure difference threshold and/or from at least one altitude difference threshold.

10. The device according to claim 5, wherein the data processing and control unit is configured to calculate at least one fluid volume value associated with each atmospheric pressure range and/or with each altitude range, the at least one fluid volume value corresponding to a subdivision of a maximum volume to be withdrawn or to be injected during an atmospheric pressure variation and/or a maximum altitude variation.

11. The device according to claim 5, wherein the data processing and control unit is configured to implement a step of:
x) detecting whether the estimate of the atmospheric pressure crosses at least one atmospheric pressure threshold or whether an estimate of an altitude crosses at least one altitude threshold, the at least one atmospheric pressure threshold being a lower or upper limit of an atmospheric pressure range and the at least one altitude threshold being a lower or upper limit of an altitude range.

12. The device according to claim 1, wherein the data processing and control unit is configured to exchange data with an external control element comprising a barometer and/or an altimeter and/or a GPS, the data processing and control unit being configured to estimate the atmospheric pressure based on atmospheric pressure measurement data of the barometer and/or to estimate the altitude based on altitude measurement data of the altimeter and/or of the GPS.

13. The device according to claim 12, wherein the data processing and control unit is configured to record the atmospheric pressure measurement by the barometer as an estimate of the atmospheric pressure if the measured atmospheric pressure is comprised between 1054 mbar and 694 mbar and/or to record the altitude measurement by the altimeter as an estimate of the altitude if the measured altitude is comprised between 0 meters and 3000 meters above sea level.

14. The device according to claim 1, wherein the data processing and control unit is configured to calculate the fluid volume to be withdrawn depending on an estimate of a second fluid volume which was injected into the inflatable element due to a deformation of the reservoir caused by a reduction of the atmospheric pressure and/or to an increase of the altitude.

15. The device according to claim 1, wherein the data processing and control unit is configured to calculate the fluid volume to be injected depending on an estimate of a second fluid volume which was withdrawn from the inflatable element due to a deformation of the reservoir 5 caused by an increase in the atmospheric pressure and/or a reduction of the altitude.

16. The device according to claim 1, wherein the data processing and control unit is configured to exchange data with an external control element comprising a barometer and/or an altimeter and/or a GPS and configured, if a command is implemented by a patient in whom the device is implanted via the external control element, to record an atmospheric pressure measurement by the barometer and/or an altitude measurement by the altimeter, and/or a measurement of the altitude by the GPS.

17. The device according to claim 1 in combination with claim 16 or according to claim 12 in combination with claims 5 and 16, wherein the data processing and control unit is configured to update the lower limit and the upper limit of the atmospheric pressure range(s) due to the recording of an atmospheric pressure measurement by the barometer and/or altitude measurement by the altimeter and/or a measurement of the altitude by the GPS, so that an atmospheric pressure range is centered on the atmospheric pressure measurement, and/or update the lower limit and the upper limit of the altitude range(s) so that an altitude range is centered on the altitude measurement.

18. The device according to claim 17, wherein the data processing and control unit is configured to increase the extent of the atmospheric pressure range centered on the atmospheric pressure measurement and/or of the altitude range centered on the altitude measurement.

19. The device according to claim 17, wherein the data processing and control unit is configured to calculate a fluid volume to be withdrawn from the inflatable element to the reservoir by means of the actuator or to inject from the reservoir to the inflatable element by means of the actuator by applying the atmospheric pressure or altitude measurement to a function, due to the recording of an atmospheric pressure measurement by the barometer and/or of an altitude measurement by the altimeter and/or an altitude measurement by the GPS.

20. The device according to claim 1, wherein the data processing and control unit is configured to exchange data with an external control element comprising a barometer and/or an altimeter to record, at a predetermined frequency and atmospheric pressure measurement by the barometer and/or an altitude measurement by the altimeter.

21. The device according to claim 1, wherein the data processing and control unit is configured to command an increase or a decrease in the volume of the reservoir by the actuator so as to allow the withdrawal or the injection of the fluid volume from the inflatable element to the reservoir.

22. The device according to claim 1, wherein the data processing and control unit is configured to implement step a) periodically and/or step b) periodically.

23. The device according to claim 1, configured to be implanted into a human or animal body to selectively block an anatomical passage of the human or animal body selected from at least one of the following passages: a urethra, a gastric passage, a colon or a rectum, or comprising an elongate shaped inflatable element configured to be used as penile implants.

24. An assembly comprising an implantable medical device according to claim 1 and an external control element suitable for exchanging data with the implantable medical device and configured to be used by an individual into whom the medical device is implanted, wherein the implantable medical device and the external control element comprise communication means suitable for communicating with one another.

25. A method for compensating an atmospheric pressure variation to which an implantable medical device is exposed, the implantable medical device comprising a variable-volume fluid reservoir able to deform under the influence of an atmospheric pressure variation, an inflatable element in fluid connection with the reservoir, an actuator suitable for selectively varying the volume of the fluid reservoir, and a data processing and control unit configured to control a selective variation of the volume of the fluid reservoir by the actuator, the method comprising at least one of the steps implemented by the data processing and control unit, if an estimate of an atmospheric pressure crosses at least one atmospheric pressure threshold or if an estimate of an altitude crosses at least one altitude threshold, of:
a) sending by the control unit of at least one order to the actuator to vary the volume of the reservoir for withdrawing a volume of fluid from the inflatable element to the reservoir;
b) sending by the control unit of at least one order to the actuator to vary the volume of the reservoir for injecting a volume of fluid from the reservoir to the inflatable element.

26. A computer program product comprising code instructions for the execution of a method according to claim 25.

27. A storage means readable by computing equipment wherein a computer program product understands code instructions for the execution of the method according to claim 25.
